(19) United States
(12) Patent Application Publication
(10) Pub. No.: US 2008/0255507 A1
(43) Pub. Date: Oct. 16, 2008

(54) Catheter Assembly Having a Grooved Distal Tip

(75) Inventor: Durgham Mushtaha, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/424,275

(22) Filed: Jun. 15, 2006

(51) Int. Cl. A61M 25/10; A61M 25/00

(52) U.S. Cl. 604/103; 604/523; 604/96.01

(57) ABSTRACT

A catheter assembly comprises an elongate shaft having a proximal end and a distal end. The assembly also comprises a flexible distal tip having a proximal end coupled to the distal end of the elongate shaft and having a distal end. The flexible distal tip has at least one groove in a surface thereof.

### BACKGROUND OF THE INVENTION

This invention relates generally to intravascular medical devices, and more particularly, to a catheter assembly including a grooved distal tip.

Intravascular diseases are commonly treated by techniques such as percutaneous translumenal angioplasty (PTA) and percutaneous translumenal coronary angioplasty (PTCA). Catheter-based treatments and diagnostic techniques may also include atherectomy, laser radiation, ultrasonic imaging, and others. Such techniques are well known and may involve the use of a catheter, such as a balloon catheter or a catheter having a different form of therapeutic device deployed near its distal end. The assembly typically includes a guidewire and may also be used in combination with other intravascular devices.

A balloon catheter includes an elongate shaft, a balloon attached near the distal end of the shaft, and a manifold attached to the proximal end of the shaft. When being used, a balloon catheter is advanced through a patient's vasculature over the guidewire to position the balloon adjacent a restriction in a diseased blood vessel. The balloon may then be inflated and the restriction in the vessel opened.

In some cases, the treatment of intravascular diseases may include the use of a balloon catheter to deploy a stent within the lumen of the diseased blood vessel at the target area. The stent is generally cylindrical having a lumen throughout that is positioned in a compressed configuration at the site of a lesion and then expanded by inflating the balloon to open the blood vessel. Stents are typically made of a metal material and generally include a pattern of interconnected struts. There are two basic types of balloon catheters that are used in conjunction with a guidewire; over-the-wire (OTW) catheters and single-operator-exchange (SOE) catheters. The construction and use of both types of catheters and the types and configuration of stents they deploy are well known to those skilled in the art.

Pushability, trackability, and crossability are characteristics that are important in the design of intravascular catheters. Pushability refers to the ability to transmit force from a proximal end of the catheter to the distal end of the catheter. Trackability refers to the ability of the catheter to navigate tortuous vasculature and is therefore dependent upon the flexibility of the catheter and the recoverability of the catheter; i.e., the ability of the catheter to bend and then return to its normal configuration after being bent. Crossability refers to the ability of the catheter to navigate through narrow restrictions in the vasculature such as a stenosis or fully and partially deployed stents.

The trackability of a catheter is generally determined by the trackability of the catheter's distal portion. This is the part of the catheter that must track the guidewire through the small tortuous vessels to reach the stenosis to be treated. A more flexible distal portion has been found to improve trackability. On the other hand, it has been found that kinking may occur when transitioning from a stiff proximal segment of the catheter shaft to a more flexible distal portion of the catheter shaft. This kinking particularly occurs at the joint between the two shaft segments of differing flexibility. An increase in the flexibility of the distal section may also make this portion of the catheter less able to be pushed from the proximal end of the catheter (i.e., reduce pushability).

Crossability is related to the flexibility of the distal section of the catheter and, in the area of a lesion, by the design of the distal tip of the catheter; i.e., the outer diameter or crossing profile of the distal tip which first contacts the inner walls of the vascular system or a target lesion to be treated. Clearly, a smaller outer diameter of the distal tip creates a smaller entry or crossing profile.

As stated previously, pushability is related to the ability of the catheter to transmit force from its proximal end to its distal end. A catheter must possess sufficient stiffness to be pushed through vessels and have sufficient rigidity to provide sufficient torsional control. However, excessive stiffness or rigidity in the catheter tip may damage the lining of a vessel as the catheter advances through the vascular system or a target lesion. For these reasons, it is desirable for a catheter to have a soft or flexible distal tip.

Thus, in view of the above-mentioned considerations it would be desirable to provide a catheter assembly having a reduced crossing profile at the distal tip of the catheter while maintaining the required pushability and trackability.

### BRIEF SUMMARY OF THE INVENTION

An improved catheter assembly is provided. The catheter assembly comprises an elongate shaft having a proximal end and a distal end, and a flexible distal tip having a proximal end coupled to the distal end of the elongate shaft and having a distal end. The flexible distal tip has at least one groove in a surface thereof.

### BRIEF DESCRIPTION OF THE DRAWINGS

The present invention will hereinafter be described in conjunction with the following drawings, wherein like reference numerals denote like elements, and

FIG. 1 is a partial cross-sectional view of a catheter assembly in accordance with the present invention;

FIG. 2 is a cross-sectional view of the distal region of the catheter assembly shown in FIG. 1;

FIG. 3 is a plan view of a catheter distal tip in accordance with a first embodiment of the present invention;

FIG. 4 is a cross-sectional view of the distal tip shown in FIG. 3 taken along line 4-4;

FIG. 5 is a cross-sectional view taken along line 5-5 in FIG. 2 illustrating how the tip seal material contacts the surface of the grooved tip assembly shown in FIG. 3; and

FIG. 6 is a plan view of a distal tip in accordance with a second embodiment of the present invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention. The drawings, which are not necessarily to scale, depict selected embodiments and are not intended to limit the scope of the invention. Examples of construction, materials, dimensions, and the manufacturing processes are provided for selected elements. All the elements employ that which is known to those skilled in the field of the invention. Those skilled in the art will recognize that many of the examples provided have suitable alternatives that may be utilized.

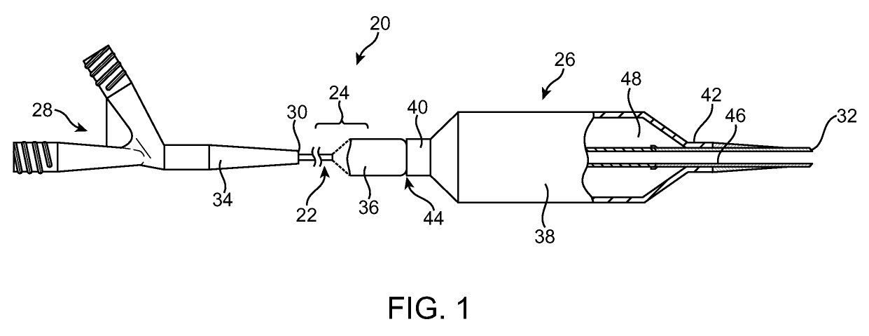

Referring now to the drawings, FIG. 1 is a cross-sectional view of an over-the-wire (OTW) balloon catheter, which is representative of one type of catheter that can incorporate the present invention. Other transvascular catheter embodiments are additionally suitable without deviating from the spirit and scope of the present invention. For example, intravascular catheters suitable for incorporating the present invention include fixed-wire (FW) catheters, single-operator-exchange (SOE) catheters, etc.

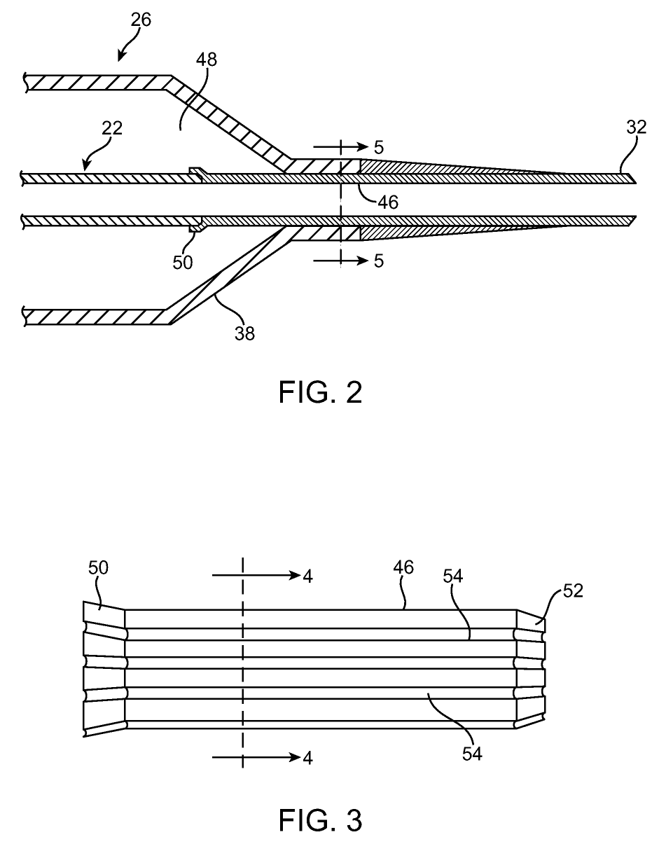

Referring to FIG. 1 described above and to FIG. 2, which is a cross-sectional view of the distal portion of the catheter shown in FIG. 1, a balloon catheter 20 includes a shaft assembly 22. It is to be noted that there is a change in scale indicated at 24 so as to facilitate a clear description of the distal portion of catheter 20. Shaft assembly 22 includes a proximal end 30 and a distal end 32. A balloon assembly 26 is coupled proximate the distal end of shaft assembly 22, and a conventional OTW-type manifold assembly 28 is coupled to the proximal end of shaft assembly 22. Proximal end 30 extends into manifold assembly 28 and is affixed thereto. A polyurethane strain relief 34 is coupled to manifold assembly 28, and shaft assembly 22 extends into manifold assembly 28 through strain relief 34. Outer tubular member 36 is coaxially disposed about an inter-tubular lumen to define an annular inflation lumen therebetween as is well known to those skilled in the art.

Materials used to form outer tubular member 36 may vary depending on the desired stiffness of shaft assembly 22. Materials suitable for use in outer tubular members include nylon and similar polyamides, polyetheretherkeytone (PEEK), polyimide (PI), and polyetherimide (PEI). Additional rigidity may be imparted to the outer tubular member 36 by incorporating a braid on or within the tubular member. Polyether block amide (PEBA), in contrast to the rigid polyamides, is a relatively flexible polyamide material having a durometer of approximately 70D.

The inner tubular member defines a guidewire lumen (not shown), which provides a passageway for the guidewire (also not shown). The inner tubular member may be made of polyethylene or, alternatively, a lubricious material such as polytetrafluouroethylene (PTFE).

Balloon assembly 26 includes a balloon body portion 38 having a proximal balloon waist 40 and a distal balloon waist 42. Proximal balloon waist 40 may be coupled to outer tubular member 36 near its distal end 44 adhesively, by thermal bonding, etc. The distal balloon waist 42 is connected to a flexible distal tip 46 by means of an adhesive or thermal bond. The interior 48 of balloon 38 is in fluid communication with the annular inflation lumen. In FIG. 2, it can be seen that distal tip 46 is provided with a flared portion 50 into which the proximal end of shaft assembly 22 is received.

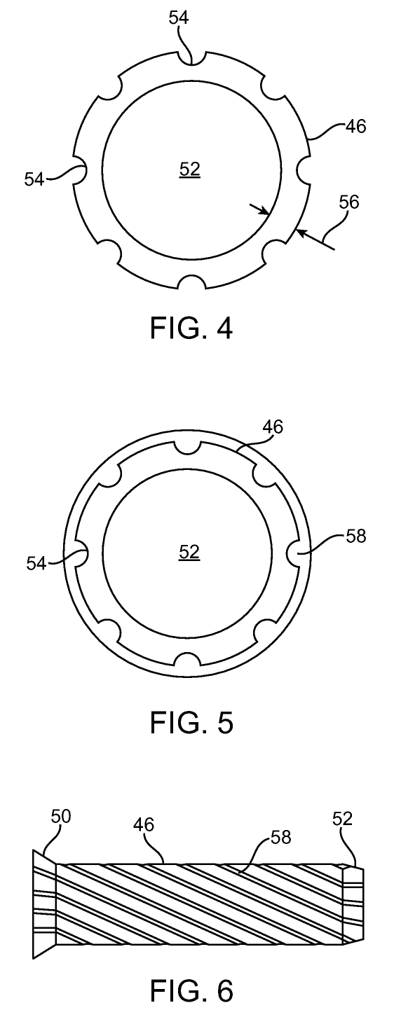

FIGS. 3 and 4 are plan and cross-sectional views, respectively, of flexible distal tip 46 in accordance with a first embodiment of the present invention. As can be seen, flexible distal tip 46 is generally cylindrical having a lumen 52 therethrough. The proximal end of distal tip 46 has a flared section 50 for receiving the distal end of shaft 22 as described above. The distal end of distal tip 46 has a portion 52 which has a progressively reduced diameter so as to present a reduced profile to the patient's vasculature. This area of a progressively reduced outer diameter may be produced by any suitable method (e.g., grinding, sanding, etc.).

As can be seen, the flexible distal tip 46 shown in FIGS. 3 and 4 is provided with at least one longitudinal groove 54 (and preferably a plurality (e.g., eight) of longitudinal groove 54) that extends along at least a portion of (and perhaps the entire length of) tip 46. Grooves 54 may be produced using well-known extrusion techniques. It should be noted that both the number and the length of grooves 54 may be varied to suit a particular application or purpose. Similarly, grooves 54 may be semi-cylindrical in shape having a depth and width of perhaps one-half the wall thickness 56; however, it should be clear that both the shape and dimensions of grooves 54 may be varied to suit a particular purpose or application. The distal waist 42 of balloon 26 is sealingly coupled to the grooved outer surface of distal tip 46. This may be accomplished adhesively or by thermal heating of at least a portion of distal waist 42 itself.

Grooves 54 provide two distinct advantages. First, since grooves 54 increase the surface area of the outer surface of distal tip 46, the sealing material contacts a greater surface area and therefore the length of the distal balloon bond can be reduced without sacrificing bond strength. Second, since some of the sealing material will occupy the interior of the grooves themselves, the amount of sealing material residing around the outer surface of the distal tip is less reducing the crossing profile at the distal bond region. This is shown in FIG. 5 wherein much of the sealing material 58 resides within grooves 54.

As stated previously, the shape and size of grooves 54 may be varied to suit a particular purpose. FIG. 6 illustrates an embodiment of the present invention wherein grooves 58 are helically disposed around the surface of flexible distal tip 46.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing an exemplary embodiment of the invention, it being understood the various changes may be made in the function and arrangement of the elements described in the exemplary embodiment without departing from the scope of the invention as set forth in the appended claims and their legal equivalents.

What is claimed is:

1. A catheter assembly, comprising:
an elongate shaft having a proximal end and a distal end; and
a flexible distal tip having a proximal end coupled to the distal end of said elongate shaft and having a distal end, said flexible distal tip having at least one groove in a surface thereof.

2. A catheter assembly according to claim 1, wherein said flexible distal tip includes a plurality of longitudinal grooves in the surface.

3. A catheter assembly according to claim 2, wherein said flexible distal tip has an inner surface and an outer surface and wherein said plurality of longitudinal grooves is in said outer surface.

4. A catheter assembly according to claim 3, wherein each of said plurality of longitudinal grooves are substantially equally spaced around said surface.

5. A catheter assembly according to claim 4, wherein said plurality of longitudinal grooves comprises eight longitudinal grooves each spaced apart by approximately forty-five degrees around said outer surface.

6. A catheter assembly according to claim 4, wherein said substantially cylindrical wall defining said flexible tip has a thickness and wherein the depth of each of said grooves is approximately one-half that of said thickness.

7. A catheter assembly according to claim 6, wherein the width of each of said grooves is approximately one-half that of said thickness.

8. A catheter assembly according to claim 4, wherein each of said plurality of longitudinal grooves extends from the proximal end of said flexible distal tip to the distal end of said flexible distal tip.

9. A catheter assembly according to claim 8, wherein the proximal end of said flexible distal tip is flared and receives therein the distal end of said elongate shaft to couple said flexible distal tip to said elongate shaft.

10. A catheter assembly according to claim 9, wherein said catheter is a balloon catheter.

11. A catheter assembly according to claim 10, wherein said balloon catheter includes a balloon comprising:
a proximal waist portion fixedly coupled to said elongate shaft;
a distal waist portion fixedly coupled to said flexible distal tip;
an inflatable portion coupled between said proximal waist portion and said distal waist portion.

12. A catheter assembly according to claim 11, wherein said distal waist is sealed to said flexible distal tip around said plurality of longitudinal grooves.

13. A catheter assembly according to claim 12, wherein at least a portion of said distal waist is melted to form seal material that surrounds a section of said flexible distal tip filling said plurality of longitudinal grooves in said section to bond said distal waist to said flexible distal tip.

14. A catheter assembly according to claim 1, wherein said flexible distal tip includes a plurality of helical grooves in the surface.

15. A balloon catheter assembly, comprising:
an elongate shaft having a proximal end and a distal end;
a distal tip having a proximal end coupled to the distal end of said elongate shaft and having a distal end, said distal tip having a grooved region including plurality of longitudinal grooves in a surface of said distal tip; and
a balloon having a distal waist sealingly coupled to the grooved region.

16. A balloon catheter assembly according to claim 15, wherein said distal waist is melted and sealingly engages said grooved region.

17. A balloon catheter assembly according to claim 15, wherein said flexible distal tip has an inner surface and an outer surface and wherein said plurality of longitudinal grooves is in said outer surface.

18. A balloon catheter assembly according to claim 18, wherein each of said plurality of longitudinal grooves are substantially equally spaced around said flexible distal tip.

19. A catheter assembly according to claim 15, wherein said plurality of longitudinal grooves comprise eight longitudinal grooves.

20. A catheter according to claim 19 wherein said plurality of longitudinal grooves extends from a proximal end of said flexible distal tip to a distal end of said flexible distal tip.
